(19) United States
(12) Patent Application Publication
(10) Pub. No.: US 2023/0338349 A1
(43) Pub. Date: Oct. 26, 2023

(54) LOW DOSE REGIMEN AND FORMULATION OF A 5-METHYL-1,2,4-OXADIAZOL-3-YL COMPOUND

(71) Applicant: Eli Lilly and Company, Indianapolis, IN (US)

(72) Inventors: William Brian KIELBASA, Orchard Park, NY (US); Dustin James MERGOTT, Indianapolis, IN (US)

(21) Appl. No.: 18/005,245

(22) PCT Filed: Jul. 23, 2021
(86) PCT No.: PCT/US2021/042877

§ 371 Date: Jan. 12, 2023

Related U.S. Application Data
(60) Provisional application No. 63/055,362, filed on Jul. 23, 2020.

(51) Int. Cl. A61K 31/4439; A61P 25/28

(52) U.S. Cl. CPC A61K 31/4439; A61P 25/28

(57) ABSTRACT

The present invention provides a compound of Formula I:

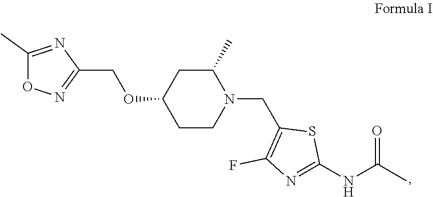 [SMILES: CC(=O)Nc1nc(F)c(CN2CC[C@H](OCc3noc(C)n3)C[C@@H]2C)s1]

or a pharmaceutically acceptable salt thereof, wherein the compound of Formula I, or pharmaceutically acceptable salt thereof is administered orally with a total dose of the compound of 0.1 mg/day to 5 mg/day, useful for treating a neurodegenerative disease, including neurodegenerative tauopathies, such as Alzheimer's disease, frontotemporal dementia, corticobasilar syndrome, and progressive supranuclear palsy.

The present invention relates to a low-dose regimen and low-dose formulation of the O-GlcNAcase (OGA) inhibitor N-[4-fluoro-5-[[(2S,4S)-2-methyl-4-[(5-methyl-1,2,4-oxadiazol-3-yl)methoxy]-1-piperidyl]methyl]thiazol-2-yl]acetamide, or pharmaceutically acceptable salt thereof, to treat neurodegenerative diseases, including, neurodegenerative tauopathies such as Alzheimer's disease (AD), frontotemporal dementia (FTD), corticobasilar syndrome (CBS), and progressive supranuclear palsy (PSP).

U.S. Pat. No. 10,081,625 discloses certain compounds, including 5-methy-1,2,4-oxadiazol-3-yl compounds, including N-[4-fluoro-5-[[(2S,4S)-2-methyl-4-[(5-methyl-1,2,4-oxadiazol-3-yl)methoxy]-1-piperidyl]methyl]thiazol-2-yl]acetamide which are OGA inhibitors useful for treating neurodegenerative diseases and disorders, such as AD and PSP.

Patients suffering from neurodegenerative diseases, including AD, PSP, and other neurodegenerative tauopathies, may develop difficulty swallowing as the disease progresses. Thus, it is desired that prescribed dosages of a drug are of a low dose providing a small unit dosage form, in order to facilitate swallowing when the drug is administered to the patient. Furthermore, in view of the progressive cognitive impairment often associated with neurodegenerative diseases such as AD, reducing the number of dosage administrations per day will help minimize the risk that a patient will miss a dose and will also reduce the burden on the caregiver responsible for ensuring proper and timely administration of the drug. In addition, lower doses and less frequent dose administration will ameliorate or eliminate potential side effects, particularly those that might arise with chronic administration (See W. Cook, et. al., 2020 Society of Toxicology Annual Meeting (virtual), Abstract No. 2911, May 12, 2020; https://www.toxicology.org/events/am/AM2020/docs/2020-ePoster-index.pdf).

Thus, low doses of OGA inhibitors are desired to provide treatment for neurodegenerative diseases, such as AD and PSP and other neurodegenerative tauopathies. In addition, administration of OGA inhibitors of no more than twice per day, and preferably once per day, are further desired for treatment of neurodegenerative diseases, including neurodegenerative tauopathies such as AD, FTD, CBS, and PSP.

Accordingly, in one embodiment, the invention provides a compound of Formula I:

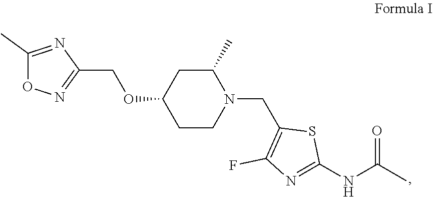 [SMILES: CC(=O)Nc1nc(F)c(CN2CC[C@H](OCc3noc(C)n3)C[C@@H]2C)s1]

or a pharmaceutically acceptable salt thereof, for use in treating a neurodegenerative disease wherein the compound, or pharmaceutically acceptable salt thereof is administered orally with a total dose of the compound of 0.1 mg/day to 5 mg/day.

In an embodiment, the invention provides a compound of Formula I:

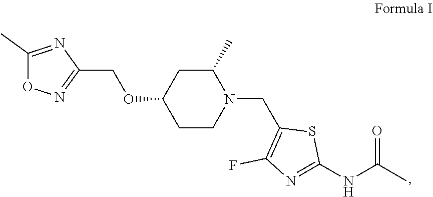 [SMILES: CC(=O)Nc1nc(F)c(CN2CC[C@H](OCc3noc(C)n3)C[C@@H]2C)s1]

or a pharmaceutically acceptable salt thereof, wherein the compound, or pharmaceutically acceptable salt thereof is administered orally with a total dose of the compound of 0.1 mg/day to 5 mg/day for use in treating Alzheimer's disease.

In an embodiment, the invention provides a compound of Formula I:

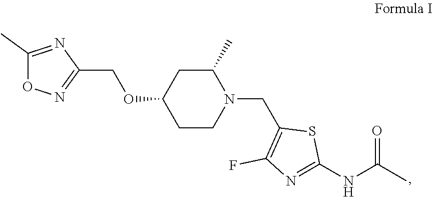 [SMILES: CC(=O)Nc1nc(F)c(CN2CC[C@H](OCc3noc(C)n3)C[C@@H]2C)s1]

or a pharmaceutically acceptable salt thereof, wherein the compound, or pharmaceutically acceptable salt thereof is administered orally with a total dose of the compound of 0.1 mg/day to 5 mg/day for use in treating progressive supranuclear palsy.

In an embodiment, the invention provides a compound of Formula I:

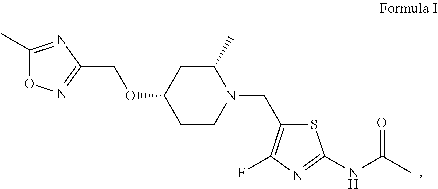 [SMILES: CC(=O)Nc1nc(F)c(CN2CC[C@H](OCc3noc(C)n3)C[C@@H]2C)s1]

or a pharmaceutically acceptable salt thereof, wherein the compound, or pharmaceutically acceptable salt thereof is administered orally with a total dose of the compound of 0.1 mg/day to 5 mg/day for use in preventing the progression of mild cognitive impairment to Alzheimer's disease.

In an embodiment, the invention provides the use of a compound of Formula I:

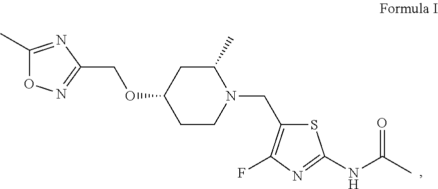 [SMILES: CC(=O)Nc1nc(F)c(CN2CC[C@H](OCc3noc(C)n3)C[C@@H]2C)s1]

or a pharmaceutically acceptable salt thereof, for the manufacture of a medicament for treating a neurodegenerative disease wherein the compound, or pharmaceutically acceptable salt thereof is administered orally with a total dose of the compound of 0.1 mg/day to 5 mg/day.

In an embodiment, the invention provides the use of a compound of Formula I:

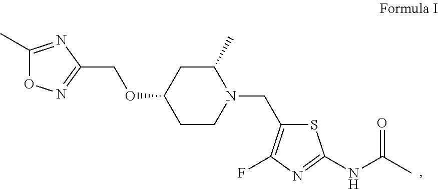 [SMILES: CC(=O)Nc1nc(F)c(CN2CC[C@H](OCc3noc(C)n3)C[C@@H]2C)s1]

or a pharmaceutically acceptable salt thereof, for the manufacture of a medicament for treating Alzheimer's disease wherein the compound, or pharmaceutically acceptable salt thereof is administered orally with a total dose of the compound of 0.1 mg/day to 5 mg/day.

In an embodiment, the invention provides a compound of Formula I:

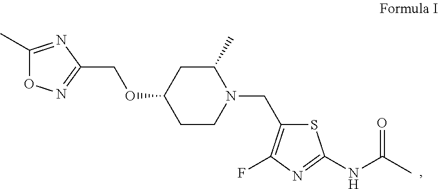 [SMILES: CC(=O)Nc1nc(F)c(CN2CC[C@H](OCc3noc(C)n3)C[C@@H]2C)s1]

or a pharmaceutically acceptable salt thereof, for the manufacture of a medicament for treating progressive supranuclear palsy wherein the compound, or pharmaceutically acceptable salt thereof is administered orally with a total dose of the compound of 0.1 mg/day to 5 mg/day.

In an embodiment, the invention provides the use of a compound of Formula I:

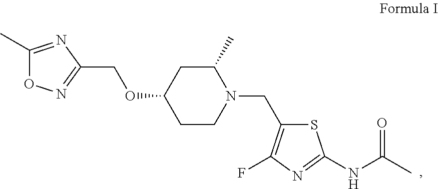 [SMILES: CC(=O)Nc1nc(F)c(CN2CC[C@H](OCc3noc(C)n3)C[C@@H]2C)s1]

or a pharmaceutically acceptable salt thereof, for the manufacture of a medicament for preventing the progression of mild cognitive impairment to Alzheimer's disease, wherein the compound, or pharmaceutically acceptable salt thereof is administered orally with a total dose of the compound of 0.1 mg/day to 5 mg/day.

In an embodiment, the invention provides a method of treating a neurodegenerative disease, comprising administering to a patient in need of such treatment a compound of Formula I:

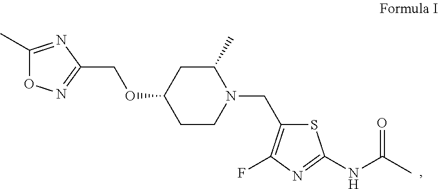 [SMILES: CC(=O)Nc1nc(F)c(CN2CC[C@H](OCc3noc(C)n3)C[C@@H]2C)s1]

or a pharmaceutically acceptable salt thereof, wherein the compound, or pharmaceutically acceptable salt thereof is administered orally with a total dose of the compound of 0.1 mg/day to 5 mg/day.

In an embodiment, the invention provides a method of treating Alzheimer's disease, comprising administering to a patient in need of such treatment a compound of Formula I:

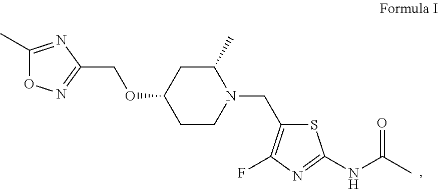 [SMILES: CC(=O)Nc1nc(F)c(CN2CC[C@H](OCc3noc(C)n3)C[C@@H]2C)s1]

or a pharmaceutically acceptable salt thereof, wherein the compound, or pharmaceutically acceptable salt thereof is administered orally with a total dose of the compound of 0.1 mg/day to 5 mg/day.

In an embodiment, the invention provides a method of treating progressive supranuclear palsy, comprising administering to a patient in need of such treatment a compound of Formula I:

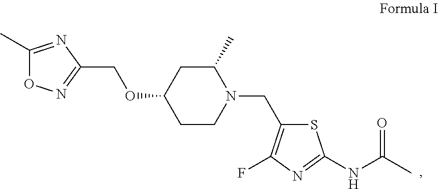 [SMILES: CC(=O)Nc1nc(F)c(CN2CC[C@H](OCc3noc(C)n3)C[C@@H]2C)s1]

or a pharmaceutically acceptable salt thereof, wherein the compound, or pharmaceutically acceptable salt thereof is administered orally with a total dose of the compound of 0.1 mg/day to 5 mg/day.

In an embodiment, the invention provides a method of preventing the progression of mild cognitive impairment to Alzheimer's disease, comprising administering to a patient in need of such treatment a compound of the of Formula I:

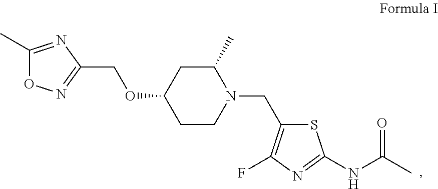 [SMILES: CC(=O)Nc1nc(F)c(CN2CC[C@H](OCc3noc(C)n3)C[C@@H]2C)s1]

or a pharmaceutically acceptable salt thereof, wherein the compound, or pharmaceutically acceptable salt thereof is administered orally with a total dose of the compound of 0.1 mg/day to 5 mg/day.

In an embodiment, the invention provides a pharmaceutical composition, comprising a compound of Formula I:

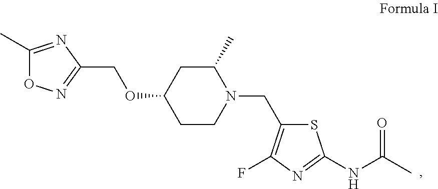 [SMILES: CC(=O)Nc1nc(F)c(CN2CC[C@H](OCc3noc(C)n3)C[C@@H]2C)s1]

or a pharmaceutically acceptable salt thereof, for use in treating a neurodegenerative disease wherein the pharmaceutical composition is administered orally with a total dose of the compound of 0.1 mg/day to 5 mg/day.

In an embodiment, the invention provides a pharmaceutical composition, comprising a compound of Formula I:

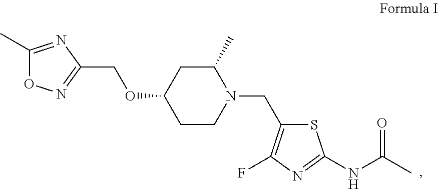 [SMILES: CC(=O)Nc1nc(F)c(CN2CC[C@H](OCc3noc(C)n3)C[C@@H]2C)s1]

or a pharmaceutically acceptable salt thereof with one or more pharmaceutically acceptable carriers, diluents, or excipients wherein the pharmaceutical composition contains a total dose of the compound of 0.1 mg to 5 mg.

In an embodiment, the invention provides a pharmaceutical composition, comprising a compound of Formula I:

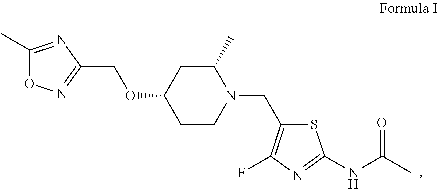 [SMILES: CC(=O)Nc1nc(F)c(CN2CC[C@H](OCc3noc(C)n3)C[C@@H]2C)s1]

or a pharmaceutically acceptable salt thereof with one or more pharmaceutically acceptable carriers, diluents, or excipients wherein the pharmaceutical composition contains a total dose of the compound of 0.1 mg to 5 mg in unit dose form wherein the unit dose is administered once per day.

In an embodiment, the invention provides a pharmaceutical composition, comprising a compound of Formula I:

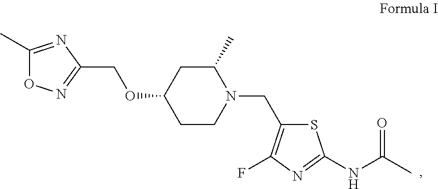 [SMILES: CC(=O)Nc1nc(F)c(CN2CC[C@H](OCc3noc(C)n3)C[C@@H]2C)s1]

or a pharmaceutically acceptable salt thereof with one or more pharmaceutically acceptable carriers, diluents, or excipients wherein the pharmaceutical composition contains a total dose of the compound of 0.05 mg to 2.5 mg in unit dose form wherein the unit dose is administered twice per day.

In an embodiment, it is understood that the compound of Formula I is in the free base form.

In a particular embodiment, the total dose of the compound of Formula I is 0.25 mg/day to 5 mg/day.

In a particular embodiment, the total dose of the compound of Formula I is 0.1 mg/day to 3 mg/day.

In a particular embodiment, the total dose of the compound of Formula I is 0.25 mg/day to 3 mg/day.

In a particular embodiment, the total dose of the compound of Formula I is 0.1 mg/day to 2 mg/day.

In a particular embodiment, the total dose of the compound of Formula I is 0.25 mg/day to 2 mg/day.

In a particular embodiment, the total dose of the compound of Formula I is 0.1 mg/day to 1 mg/day.

In a particular embodiment, the total dose of the compound of Formula I is 0.25 mg/day to 1 mg/day.

In a particular embodiment, the total dose of the compound of Formula I is 5 mg/day.

In a particular embodiment, the total dose of the compound of Formula I is 3 mg/day.

In a particular embodiment, the total dose of the compound of Formula I is 2.5 mg/day.

In a particular embodiment, the total dose of the compound of Formula I is 2 mg/day.

In a particular embodiment, the total dose of the compound of Formula I is 1.5 mg/day.

In a particular embodiment, the total dose of the compound of Formula I is 1 mg/day.

In a particular embodiment, the total dose of the compound of Formula I is 0.75 mg/day.

In a particular embodiment, the total dose of the compound of Formula I is 0.5 mg/day.

In a particular embodiment, the total dose of the compound of Formula I is 0.25 mg/day.

In a particular embodiment, the total dose of the compound of Formula I is 0.1 mg/day.

In a particular embodiment, the compound of Formula I or pharmaceutically acceptable salt thereof is administered in unit dosage form.

In a particular embodiment, the unit dosage form is selected from a capsule, tablet, gelcap, liquid solution, and liquid suspension.

In a particular embodiment, the unit dosage form is a capsule.

In a particular embodiment, the unit dosage form is a tablet.

In a particular embodiment, the total daily dose of the compound of Formula I is administered in one unit dose.

In a particular embodiment, the total daily dose of the compound of Formula I is administered in two unit doses.

In a particular embodiment, the total daily dose of the compound of Formula I is administered in two unit doses each containing equal amounts of the compound of Formula I.

In a particular embodiment, the total daily dose of the compound of Formula I is in two unit doses wherein the administration of each unit dose is separated by at least 8 hours.

In a particular embodiment, the total daily dose of the compound of Formula I is in two unit doses each containing equal amounts of the compound of Formula I wherein the administration of each unit dose is separated by at least 8 hours.

Mild cognitive impairment has been defined as a potential prodromal phase of dementia associated with Alzheimer's disease based on clinical presentation and on progression of patients exhibiting mild cognitive impairment to Alzheimer's dementia over time. The term “preventing the progression of mild cognitive impairment to Alzheimer's disease” includes restraining, slowing, stopping, or reversing the progression of mild cognitive impairment to Alzheimer's disease in a patient.

As used herein, the terms “treating” or “to treat” includes restraining, slowing, stopping, or reversing the progression or severity of an existing symptom, disease, or disorder.

As used herein, the term “patient” refers to a human.

The compounds of the present invention are preferably formulated as pharmaceutical compositions for oral administration. Such pharmaceutical compositions and processes for preparing same are well known in the art (See, e.g., Remington: The Science and Practice of Pharmacy, L. V. Allen, Editor, 22^{nd} Edition, Pharmaceutical Press, 2012).

The compound of Formula I, or pharmaceutically acceptable salts thereof, may be prepared by a variety of procedures known to one of ordinary skill in the art, for example following the procedures disclosed in U.S. Pat. No. 10,081,625.

The compound of Formula I is also known as N-[4-fluoro-5-[[(2S,4S)-2-methyl-4-[(5-methyl-1,2,4-oxadiazol-3-yl)methoxy]-1-piperidyl]methyl]thiazol-2-yl]acetamide.

### A Single Ascending Dose Study in Healthy Subjects to Assess the Safety and Pharmacokinetics of the Compound of Formula I

A Phase 1, single-center, subject- and investigator-blind, single-ascending dose, placebo-controlled, crossover, randomized study is performed to evaluate safety, tolerability, and pharmacokinetics (PK) of the compound of Formula I in healthy subjects. The study is conducted in 2 alternating cohorts (cohorts 1 and 2) in up to 3 study periods across 6 dose levels. Subjects are randomized to 1 of 3 treatment sequences in each cohort, with each sequence including 2 doses of compound of Formula I and 1 placebo dose over the 3 study periods in a complete crossover manner. The clinical study design is summarized in Table 1.

TABLE 1
Clinical Study Design

| | Cohort 1 | Cohort 2 |
|---|---|---|
| Period 1 | 0.15 mg compound of | — |
| | Formula I or placebo | |
| Period 1 | ≥7 days wash-out | 0.6 mg compound of |
| | | Formula I or placebo |
| Period 2 | 2 mg compound of | ≥7 days wash-out |
| | Formula I or placebo | |
| Period 2 | ≥7 days wash-out | 5 mg compound of |
| | | Formula I or placebo |
| Period 3 | 10 mg compound of | ≥7 days wash-out |
| | Formula I or placebo | |
| Period 3 | — | 16 mg compound of |
| | | Formula I or placebo |

After an overnight fast of at least 8 hours, oral capsules are administered with approximately 240 mL of room-temperature water in the morning of each dosing day in a sitting position. Doses of 0.15 mg to 2 mg are administered as an oral solution of compound of Formula I via an oral dosing syringe with water similar to oral capsule dosing. Tables 2a and 2b summarize the treatment regimens.

TABLE 2a
Treatment Regimens for Compound of Formula I.

| | | |
|---|---|---|
| Dosage Strength (mg) | 0.15 to 2 | 5 to 16 |
| Dosage formulation | Oral solution | Capsule |
| Dosage | Extemporaneous | Extemporaneous |
| | preparation | preparation |
| Route of administration | Oral | Oral |
| Dosing instructions | Single dose | Single dose |

TABLE 2b
Treatment Regimens for Placebo.

| | | |
|---|---|---|
| Dosage Strength (mg) | Not applicable | Not applicable |
| Dosage formulation | Oral solution (vehicle) | Capsule (HPMC¹) |
| Dosage | Matching placebo | Matching placebo |
| Route of administration | Oral | Oral |
| Dosing instructions | Single dose | Single dose |

¹HPMC = hypromellose

The capsules containing the compound of Formula I are prepared extemporaneously. Oral doses of 0.15 mg to 2 mg of the compound of Formula I are prepared extemporaneously as drug in solution. For any specific cohort/dosing period, the total number of capsules administered is the same for all subjects, regardless of whether assigned to placebo or the compound of Formula I. However, the number of capsules may vary between dosing periods and cohorts. This is similarly the case for extemporaneously prepared oral dosing solutions to maintain the blind. The compound of Formula I is supplied in the form of free base with no inactive ingredients for extemporaneous preparation. A matching volume of oral solution vehicle without compound of Formula I is used as placebo for the doses of 0.15 mg to 2 mg.

Venous blood samples of approximately 3 mL each are collected to determine the plasma concentrations of compound of Formula I. Concentrations of compound of Formula I are assayed using a validated liquid chromatography with tandem mass spectrometry method. PK parameter estimates for compound of Formula I are calculated using standard noncompartmental methods of analysis. Plasma concentrations of compound of Formula I are summarized by administered dose of compound of Formula I and the approximate time of PK blood sample collection. Following the procedure essentially as described above, PK data for a single ascending dose of the compound of Formula I in healthy subjects are set forth in Table 3 and Table 4.

TABLE 3
Mean plasma concentrations for a single ascending dose study following
oral administration of the compound of Formula I in healthy subjects.

| Dose (mg) | | | | | | |
|---|---|---|---|---|---|---|
| Compound of | | | | | | |
| Formula I | 0.15 | 0.6 | 2 | 5 | 10 | 16 |
| Time (h) | Compound of Formula I Plasma Concentration (ng/mL) | | | | | |
| 0.5 | 0.271 | 6.15 | 16.2 | 14.6 | 28.8 | 59.7 |
| 1 | 0.278 | 6.25 | 21.8 | 58.9 | 122 | 150 |
| 1.5 | 0.246 | 5.21 | 19.5 | 55.3 | 127 | 183 |
| 2 | 0.179 | 4.84 | 18.6 | 50.3 | 125 | 187 |
| 3 | 0.161 | 3.96 | 15.3 | 43.1 | 106 | 153 |
| 4 | . . . | 3.6 | 12.6 | 36.5 | 100 | 127 |
| 5 | . . . | 3.02 | 10.5 | 29.5 | 81.7 | 100 |
| 6 | . . . | 2.35 | 8.75 | 24.8 | 72.3 | 88 |
| 8 | . . . | 2.1 | 7.09 | 18.4 | 55.1 | 63 |
| 12 | . . . | 1.09 | 4.24 | 9.74 | 32.7 | 32.2 |
| 24 | . . . | 0.307 | 1.34 | 2.76 | 11 | 6.83 |
| 48 | . . . | . . . | . . . | 0.634 | 2.28 | 0.828 |

TABLE 4
PK parameters of compound of Formula I following oral administration in
healthy subjects¹.

| Dose (mg)² | 0.15 mg | 0.6 mg | 2 mg | 5 mg | 10 mg | 16 mg |
|---|---|---|---|---|---|---|
| N³ | 8 | 7 | 6 | 7 | 6 | 5 |
| CL/F⁴ | NC¹⁴ | 13.6 (33) | 15.1 (88) | 12.6 (39) | 8.20 (28) | 11.9 (19) |
| (L/h) | | | | | | |
| AUC(0-∞)⁵ | NC¹⁴ | 44.0 (33) | 133 (88) | 396 (39) | 1220 (28) | 1340 (19) |
| (ngxh/mL) | | | | | | |
| Cmax⁶ | 0.318 (52) | 6.61 (30) | 20.9 (38) | 59.1 (18) | 134 (17) | 199 (25) |
| (ng/ml) | | | | | | |
| V_{z}/F⁷ | NC¹⁴ | 120 (13) | 119 (40) | 114 (25) | 104 (21) | 102 (23) |
| (L) | | | | | | |
| t_{1/2}⁸ | NC¹⁴ | 6.08 | 5.48 | 6.23 | 8.82 | 5.92 |
| (h) | | (4.35-9.76) | (3.94-11.2) | (3.82-8.48) | (6.91-12.4) | (4.40-7.33) |
| tₘₐₓ⁹ | 1.02 | 1.00 | 1.25 | 1.02 | 1.50 | 1.98 |
| (h) | (0.50-1.50) | (0.50-1.50) | (1.00-2.00) | (1.00-3.00) | (1.00-2.07) | (1.00-2.00) |
| Ae0-24¹⁰ | 0.003 | 0.048 (53%) | 0.155 (57%) | 0.436 (57%) | 1.3 (30%) | 1.14 (36%) |
| (mg) | (52%)¹² | | | | | |
| CLr¹¹ | NC¹⁴ | 1.17 (30) | 1.25 (30) | 1.18 (32) | 1.22 (10) | 0.96 (46)¹³ |
| (L/h) | | | | | | |

¹Data presented as geometric mean (% CV geometric mean) unless noted otherwise
²Amount of compound of Formula I administered
³N = number of subjects
⁴CL/F = apparent clearance calculated after oral administration
⁵AUC(0-∞) = area under the concentration versus time curve from time zero to infinity
⁶Cmax = maximum observed drug concentration
⁷V_{z}/F = apparent volume of distribution after oral administration
⁸t_{1/2} = terminal half-life (geometric mean (min-max))
⁹tₘₐₓ = time of maximum observed drug concentration; median (min-max)
¹⁰Ae0-24 = amount of compound of Formula I excreted in urine up to 24 hours postdose
¹¹CLr = renal clearance
¹²N=5
¹³N = 3
¹⁴NC = not calculated

The data discloses that the AUC(0-∞) and Cₘₐₓ increase approximately dose-proportionally over the 0.6-mg to 16-mg dose range for the compound of Formula I. The median tₘₐₓ is about 1 hour and t_{1/2} is about 6 hours for the compound of Formula I.

### Assessment of Brain O-GlcNAcase Enzyme Occupancy after Single Oral Doses of the Compound of Formula I as Measured by Positron Emission Tomography with the Radioligand [¹⁸F]LSN3316612 in Healthy Subjects

A single-center, open-label, nonrandomized, positron emission tomography (PET) study demonstrating brain penetration and target engagement of brain O-GlcNAcase (OGA) after single oral doses of 0.25 mg, 1 mg, and 5 mg of N-[4-fluoro-5-[[(2S,4S)-2-methyl-4-[(5-methyl-1,2,4-oxadiazol-3-yl)methoxy]-1-piperidyl]methyl]thiazol-2-yl]acetamide (compound of Formula I) is carried out by one of ordinary skill in the art essentially as set forth below. [¹⁸F]LSN3316612 is a positron-emitting radiopharmaceutical for in vivo imaging of OGA in the brain and is used to evaluate target engagement of compounds which inhibit OGA. The preparation and use of ¹⁸F-LSN3316612 as a PET radioligand is known in the art, for example as described by S. Lu, et. al, Science Translational Medicine, 12, eaau2939 (2020) 13 May 2020. This single-dose PET study using the [¹⁸F]LSN3316612 tracer assesses the brain OGA enzyme occupancy (EO) across a suitable range of doses that have been demonstrated to be safe and well tolerated.

Healthy subjects are assigned to 1 of 4 cohorts with 4 subjects in each cohort completing the study. All subjects undergo one baseline PET scan and two post-dose PET scans. A baseline PET scan is performed from up to approximately 14 days before dosing of the compound of Formula I. Overall, each subject receives a single dose of the compound of Formula I and 3 administrations of the [¹⁸F]LSN3316612 PET tracer. Dosing of the compound of Formula I occurs after completion of the baseline PET scan. Scans are conducted at approximately 2 and 24 hours post-dose for the 0.25 mg and 5 mg doses of the compound of Formula I, and at approximately 2 and 24 hours or 30 and 54 hours post-dose for the 1 mg dose of the compound of Formula I. Dynamic PET data of the brain are acquired over 120 min immediately following tracer injection. EO is summarized by the compound of Formula I dose and approximate scanning time.

The compound of Formula I is administered orally in capsule formulation for doses≥3 mg. For doses lower than 3 mg, the compound of Formula I is weighed into a suitable container and dissolved in an appropriate volume of degassed Sprite® or diluent.

[¹⁸F]LSN3316612 is produced in the clinical site radiochemistry facility from the nonradioactive precursor on the day of each PET scan. [¹⁸F]LSN3316612 injection is a clear solution for intravenous injection formulated in normal saline containing ethanol, sterile water for injection, and sodium ascorbate. [¹⁸F]LSN3316612 is delivered in normal saline (0.9% NaCl) formulated with the intent to contain approximately 3.3% (v/v) ethanol (EtOH) and sodium ascorbate (4.67 mg/mL). [¹⁸F]LSN3316612 is administered intravenously over a 3-minute infusion period using an infusion pump followed by a 10-mL saline flush. Prior to the PET imaging, subjects have an intravenous catheter (for radiotracer infusion) inserted according to standard clinical practice. Each subject receives a single injection of [¹⁸F]LSN3316612 at each imaging visit. The radiopharmaceutical is injected intravenously at a dose of approximately 5 mCi (not more than 6 mCi), with a maximum mass dose of 10 μg and maximum volume of 10 mL.

For each PET scan, the radiochemistry laboratory synthesizes the radioligand from the precursor according to PET unit production protocol known in the art, such as that described by Lee, J., Liow, J., Paul, S. et al PET quantification of brain O-GlcNAcase with [¹⁸F]LSN3316612 in healthy human volunteers. EJNMNI Res 10, 20 (2020). https://doi.org/10.1186/s13550-020-0616-4.

Arterial blood samples are collected from all subjects during each PET scan to measure radioactivity to provide input for the PET tracer kinetic analysis. Venous blood samples are collected following dosing of compound of Formula I to measure plasma concentrations of compound of Formula I using a validated liquid chromatography with tandem mass spectrometry assay.

The primary imaging outcome for the [¹⁸F]LSN3316612 PET is the total distribution volume (V_{T}) which is determined in regions where OGA is present, including cortical regions, regions of basal ganglia, thalamus, and cerebellum. Analysis uses the decay-corrected time activity data in the different brain regions. Imaging data is analyzed with 2-tissue compartment model with arterial input function to determine V_{T}. OGA EO after a single dose of the compound of Formula I is obtained using graphical analysis according to the occupancy plot:

V_{T}(Baseline)−V_{T}(Dosing)=Occupancy*(V_{T}(Baseline)−V_{ND}),

where V_{T}(Baseline) and V_{T} (Dosing) are the total distribution volumes in several regions obtained at baseline and after compound of Formula I administration, respectively. The occupancy is determined as the slope of the linear regression of the plot, and the nondisplaceable volume of distribution V_{ND} as the x-intercept.

Following the procedure essentially as described above, the target engagement of brain OGA after single oral doses of 0.25 mg, 1 mg, and 5 mg of compound of Formula I is set forth in Tables 5a-5c.

TABLE 5a
Brain OGA Occupancy for the 0.25
mg dose of Compound of Formula I.

| Approximate | Mean Enzyme | Mean Plasma Concentration |
|---|---|---|
| Time | Occupancy | of Compound of Formula I |
| (h) | (%) | (ng/mL) |
| 0.5 | | 0.934 |
| 1 | | 0.595 |
| 2 | 25.6 | 0.373 |
| 3 | | 0.391 |
| 4 | | 0.367 |
| 5 | | 0.39 |
| 6 | | 0.357 |
| 8 | | 0.294 |
| 12 | | 0.213 |
| 24 | 46 | 0.107 |

TABLE 5b
Brain OGA Occupancy for the 1 mg
dose of Compound of Formula I.

| Approximate | Mean Enzyme | Mean Plasma Concentration |
|---|---|---|
| Time | Occupancy | of Compound of Formula I |
| (h) | (%) | (ng/mL) |
| 0.5 | | 6.29 |
| 1 | | 6.24 |
| 2 | 97.3 | 5.75 |
| 3 | | 4.97 |
| 4 | | 4.28 |
| 5 | | 3.87 |
| 6 | | 3.08 |
| 8 | | 2.45 |
| 12 | | 1.42 |
| 24 | 80.6 | 0.412 |
| 30 | 68 | 0.222 |
| 54 | 30.3 | 0.0735 |

TABLE 5c
Brain OGA Occupancy for the 5 mg
dose of Compound of Formula I.

| Approximate | Mean Enzyme | Mean Plasma Concentration |
|---|---|---|
| Time | Occupancy | of Compound of Formula I |
| (h) | (%) | (ng/mL) |
| 0.5 | | 32.4 |
| 1 | | 30.7 |
| 2 | 98.2 | 36 |
| 3 | | 31.9 |
| 4 | | 31.5 |
| 5 | | 30.6 |
| 6 | | 25.3 |
| 8 | | 17.8 |
| 12 | | 12.5 |
| 24 | 92.5 | 3.07 |

The data provided in Tables 5a-5c above discloses a plasma concentration-dependent change in brain OGA EO with EO for the 5 mg dose of the compound of Formula I exceeding 90% EO at 24 hours post dose. The 1 mg dose of the compound of Formula I was found to be 80.600 EO at 24 hours post dose and 30.300 EO at 54 hours post dose. The 0.25 mg dose of the compound of Formula I was found to be 46% EO at 24 hours post dose.

Utilizing the PK data from the single ascending dose study of the compound of Formula I in healthy subjects and the PET study demonstrating brain penetration and target engagement of brain OGA after single oral doses of the compound of Formula I as disclosed above, the low doses and dosage regimens for treating a neurodegenerative disease, including AD, PSP, FTD, CBS, and other neurodegenerative tauopathies, with a compound of Formula I are set forth below:
A total dose of the compound of Formula I of 0.25 mg/day to 5 mg/day.
A total dose of the compound of Formula I of 0.1 mg/day to 3 mg/day.
A total dose of the compound of Formula I of 0.25 mg/day to 3 mg/day.
A total dose of the compound of Formula I of 0.1 mg/day to 2 mg/day.
A total dose of the compound of Formula I of 0.25 mg/day to 2 mg/day.
A total dose of the compound of Formula I of 0.1 mg/day to 1 mg/day.
A total dose of the compound of Formula I of 0.25 mg/day to 1 mg/day.
A total dose of the compound of Formula I of 5 mg/day.
A total dose of the compound of Formula I of 3 mg/day.
A total dose of the compound of Formula I of 2.5 mg/day.
A total dose of the compound of Formula I of 2 mg/day.
A total dose of the compound of Formula I of 1.5 mg/day.
A total dose of the compound of Formula I of 1 mg/day.
A total dose of the compound of Formula I of 0.75 mg/day.
A total dose of the compound of Formula I of 0.5 mg/day.
A total dose is the compound of Formula I of 0.25 mg/day.
A total dose of the compound of Formula I of 0.1 mg/day.

It is preferred that the total daily dose of compound of Formula I administered is in one unit dose.

It is further preferred that the total daily dose of the compound of Formula I administered is in two unit doses. It is preferred that the total daily dose of the compound of Formula I is administered in two unit doses wherein each dose contains equal amounts of the compound of Formula I. It is preferred that when the total daily dose of the 30 compound of Formula I is administered in two unit doses, the administration of each unit dose is separated by at least 8 hours.

In addition, total doses of the compound of Formula I in a pharmaceutical composition are set forth below:
A total dose of the compound of Formula I is 0.25 mg to 5 mg.
A total dose of the compound of Formula I is 0.1 mg to 3 mg.
A total dose of the compound of Formula I is 0.25 mg to 3 mg.
A total dose of the compound of Formula I is 0.1 mg to 2 mg.
A total dose of the compound of Formula I is 0.25 mg to 2 mg.
A total dose of the compound of Formula I is 0.1 mg to 1 mg.
A total dose of the compound of Formula I is 0.25 mg to 1 mg.
A total dose of the compound of Formula I is 3 mg.
A total dose of the compound of Formula I of 2.5 mg/day.
A total dose of the compound of Formula I is 2 mg.
A total dose of the compound of Formula I is 1.5 mg.
A total dose of the compound of Formula I is 1 mg.
A total dose of the compound of Formula I is 0.75 mg.
A total dose of the compound of Formula I of 0.5 mg/day.
A total dose of the compound of Formula I is 0.25 mg.

In addition, it is preferred that the total dose of the pharmaceutical composition comprising the compound of Formula I, or pharmaceutically acceptable salt thereof is contained in one unit dose.

It is further preferred that the total dose of the pharmaceutical composition comprising the compound of Formula I, or pharmaceutically acceptable salt thereof is contained in one unit dose wherein the single unit dose is administered once per day.

It is also preferred that the total dose of the pharmaceutical composition comprising the compound of Formula I, or pharmaceutically acceptable salt thereof is contained in two unit doses.

It is preferred that the total dose of the pharmaceutical composition comprising the compound of Formula I, or pharmaceutically acceptable salt thereof is contained in two unit doses with each unit dose containing equal amounts of the compound of Formula I.

It is further preferred that the total dose of the pharmaceutical composition comprising the compound of Formula I, or pharmaceutically acceptable salt thereof is contained in two unit doses wherein each dose is administered in one day.

It is further preferred that the total dose of the pharmaceutical composition comprising the compound of Formula I, or pharmaceutically acceptable salt thereof is contained in two unit doses with each unit dose containing equal amounts of the compound of Formula I wherein each dose is administered within one day, preferably separated by at least 8 hours.

What is claimed is:

1-26. (canceled)

27. A method of treating a neurodegenerative disease, comprising administering to a patient in need of such treatment a compound of the formula: 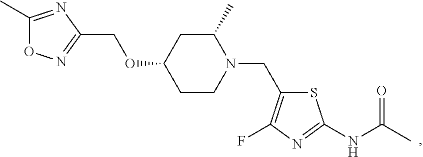 [SMILES: CC(=O)Nc1nc(F)c(CN2CC[C@H](OCc3noc(C)n3)C[C@@H]2C)s1]
or a pharmaceutically acceptable salt thereof, wherein the compound or pharmaceutically acceptable salt thereof is administered orally with a total dose of the compound of 0.1 mg/day to 5 mg/day.

28. The method according to claim 27 wherein the neurodegenerative disease is Alzheimer's disease.

29. The method according to claim 27 wherein the neurodegenerative disease is progressive supranuclear palsey.

30. The method according to claim 27, wherein the total dose of the compound is 0.25 mg/day to 5 mg/day, or 0.25 mg/day to 3 mg/day, or 0.25 mg/day to 2 mg/day, or 0.25 mg/day to 1 mg/day.

31. The method according to claim 27, wherein the total dose of the compound is 0.1 mg/day to 3 mg/day, or 0.1 mg/day to 2 mg/day, or 0.1 mg/day to 1 mg/day.

32.-36. (canceled)

37. The method according to claim 27, wherein the total dose of the compound is 3 mg/day.

38. The method according to claim 27, wherein the total dose of the compound is 2.5 mg/day.

39. The method according to claim 27, wherein the total dose of the compound is 2 mg/day.

40. The method according to claim 27, wherein the total dose of the compound is 1.5 mg/day.

41. The method according to claim 27, wherein the total dose of the compound is 1 mg/day.

42. The method according to claim 27, wherein the total dose of the compound is 0.75 mg/day.

43. The method according to claim 27, wherein the total dose of the compound is 0.5 mg/day.

44. The method according to claim 27, wherein the total dose of the compound is 0.25 mg/day.

45. The method according to claim 27, wherein the total daily dose is administered in one unit dose.

46. The method according to claim 27, wherein the total daily dose is administered in two unit doses.

47. The method according to claim 46 wherein the administration of each unit dose is separated by at least 8 hours.

48.-66. (canceled)

67. A pharmaceutical composition, comprising a compound of the formula: 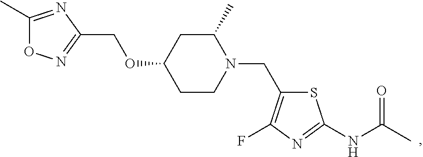 [SMILES: CC(=O)Nc1nc(F)c(CN2CC[C@H](OCc3noc(C)n3)C[C@@H]2C)s1]
or a pharmaceutically acceptable salt thereof with one or more pharmaceutically acceptable carriers, diluents, or excipients wherein the pharmaceutical composition contains a total dose of the compound of 0.1 mg to 5 mg, or 0.25 mg to 5 mg, or 0.1 mg to 3 mg, or 0.25 mg to 3 mg, or 0.1 mg to 2 mg, or 0.25 mg to 2 mg, or 0.1 mg to 1 mg, or 0.25 mg to 1 mg.

68.-74. (canceled)

75. The pharmaceutical composition according to claim 67 wherein the total dose of the compound is 3 mg.

76. The pharmaceutical composition according to claim 67 wherein the total dose of the compound is 2.5 mg.

77. The pharmaceutical composition according to claim 67 wherein the total dose of the compound is 2 mg.

78. The pharmaceutical composition according to claim 67 wherein the total dose of the compound is 1.5 mg.

79. The pharmaceutical composition according to claim 67 wherein the total dose of the compound is 1 mg.

80. The pharmaceutical composition according to claim 67 wherein the total dose of the compound is 0.75 mg.

81. The pharmaceutical composition according to claim 67 wherein the total dose of the compound is 0.5 mg.

82. The pharmaceutical composition according to claim 67 wherein the total dose of the compound is 0.25 mg.

83. The pharmaceutical composition according to claim 67 wherein the total dose of the compound is contained in one unit dose.

84. The pharmaceutical composition according to claim 67 wherein the total dose of the compound is contained in two unit doses.

85. The pharmaceutical composition according to any one of claim 67 wherein the total dose of the compound is contained in two unit doses with each unit dose containing equal amounts of the compound.

86. The pharmaceutical composition according to claim 83 wherein the one unit dose is administered once per day.

87.-90. (canceled)
